(19) United States
(12) Patent Application Publication
(10) Pub. No.: US 2025/0084021 A1
(43) Pub. Date: Mar. 13, 2025

(54) PROCESS FOR PREPARING AN ALCOHOL USING A SURFACE-REACTED CALCIUM CARBONATE CATALYST

(71) Applicant: Omya International AG, Oftringen (CH)

(72) Inventors: Jamal FTOUNI, Zofingen (CH); Jose THARUN, Utrecht (NL); Pieter C.A. BRUIJNINCX, Utrecht (NL)

(73) Assignee: Omya International AG, Oftringen (CH)

(21) Appl. No.: 18/566,345

(22) PCT Filed: Jul. 5, 2022
(86) PCT No.: PCT/EP2022/068503

§ 371 Date: Dec. 1, 2023

(30) Foreign Application Priority Data
Jul. 12, 2021 (EP) 21185121.7

(51) Int. Cl. C07C 29/34; B01J 27/232; B01J 35/40; B01J 35/61; B01J 35/63

(52) U.S. Cl. CPC C07C 29/34; B01J 27/232; B01J 35/40; B01J 35/613; B01J 35/615; B01J 35/633; B01J 35/635; B01J 35/638

(57) ABSTRACT

A process for preparing an alcohol by a Guerbet self-condensation reaction in the gas phase. The process comprises the steps of: a) providing a primary or secondary alcohol, wherein the primary or secondary alcohol has at least one β-hydrogen; b) providing a surface-reacted calcium carbonate, wherein the surface-reacted calcium carbonate is a reaction product of ground natural calcium carbonate (GNCC) or precipitated calcium carbonate (PCC) with carbon dioxide and one or more H₃O⁺ ion donors and wherein the carbon dioxide is formed in situ by the H₃O⁺ ion donors treatment and/or is supplied from an external source, and wherein the surface-reacted calcium carbonate has a specific surface area of at least 15 m²/g, measured using nitrogen and the BET method according to ISO 9277:2010; c) vaporizing the alcohol; d) reacting the vaporized alcohol in the presence of the surface-reacted calcium carbonate as a catalyst.

The present invention relates to a process for preparing an alcohol by a Guerbet self-condensation reaction in the gas phase. Further, the present invention relates to the use of a surface-reacted calcium carbonate as a catalyst for a Guerbet self-condensation reaction in the gas phase.

### BACKGROUND

Self-condensation of alcohols in the presence of a catalyst into dimerized alcohols or higher order alcohols, also known in the art as the Guerbet reaction, is an attractive process to convert comparatively simple and inexpensive alcohols into more valuable products. Different homogeneous catalysts, heterogeneous catalysts, or combinations thereof are known in the art to catalyze Guerbet reactions. For example, it is known that Guerbet reactions can be catalyzed by transition metal catalysts based on palladium, platinum, iridium, copper, nickel, ruthenium, cobalt etc. A common drawback of transition metal catalysts is that they are usually comparatively expensive and in some cases involve difficult syntheses. In addition, known catalytically systems for Guerbet reactions often need a co-catalyst to work efficiently which may complicate reaction control. Further, homogeneous catalysts are only applicable in liquid phase reactions. However, in many cases, gas phase reactions are preferred over liquid phase reactions e.g. in order to reduce waste or simplify purification of product streams.

Heterogeneous alkaline earth catalysts are useful for gas phase or vapor phase Guerbet reactions, and may avoid some of the above mentioned drawbacks associated with other catalytic systems. However, known heterogeneous alkaline earth metal catalysts often provide a rather low conversion of alcohol substrate and are limited in terms of product selectivity as well as yield thereof. Further, some known heterogeneous alkaline earth metal catalysts such as hydroxylapatite (HAP) catalysts have to be prepared by comparatively complex sol-gel processes which can make them rather costly, and may render their application unattractive from a business standpoint.

An object of the present invention is to provide an alternative or improved process for preparing an alcohol by a Guerbet self-condensation reaction in the gas phase. Another object of the present invention is to provide an alternative or improved catalyst which is useful for a Guerbet self-condensation reaction in the gas phase.

### SUMMARY OF THE INVENTION

The objects of the present invention have been achieved by the process and the use according to the independent claims.

In one aspect, the present invention provides a process for preparing an alcohol by a Guerbet self-condensation reaction in the gas phase. The process according to the invention comprises the steps of:
a) providing a primary or secondary alcohol, wherein the primary or secondary alcohol has at least one β-hydrogen;
b) providing a surface-reacted calcium carbonate, wherein the surface-reacted calcium carbonate is a reaction product of ground natural calcium carbonate (GNCC) or precipitated calcium carbonate (PCC) with carbon dioxide and one or more H₃O⁺ ion donors and wherein the carbon dioxide is formed in situ by the H₃O⁺ ion donors treatment and/or is supplied from an external source, and wherein the surface-reacted calcium carbonate has a specific surface area of at least 15 m²/g, measured using nitrogen and the BET method according to ISO 9277:2010;
c) vaporizing the alcohol provided in step a);
d) reacting the vaporized alcohol obtained in step c) in the presence of the surface-reacted calcium carbonate provided in step b) as a catalyst.

In another aspect, the present invention relates to the use a surface-reacted calcium carbonate as a catalyst for a Guerbet self-condensation reaction in the gas phase,
wherein the surface-reacted calcium carbonate is a reaction product of ground natural calcium carbonate (GNCC) or precipitated calcium carbonate (PCC) with carbon dioxide and one or more H₃O⁺ ion donors and wherein the carbon dioxide is formed in situ by the H₃O⁺ ion donors treatment and/or is supplied from an external source, and wherein the surface-reacted calcium carbonate has a specific surface area of at least 15 m²/g, measured using nitrogen and the BET method according to ISO 9277:2010.

The inventors unexpectedly found that the specific surface-reacted calcium carbonate as described herein is useful as a catalyst for self-condensing alcohols in the gas phase. The process using the specific surface-reacted calcium carbonate as catalyst provides similar or better results (conversion, selectivity, stability) compared to known alkaline earth metal catalysts for the same purpose. The process does not require a co-catalyst. Further, the surface-reacted calcium carbonate can be obtained by surface reacting ground natural calcium carbonate or precipitated calcium carbonate as described herein, and is therefore obtainable from readily available and comparatively cheap starting materials.

Preferred embodiments of the present invention are defined in the dependent claims.

According to one embodiment of the present invention, the primary or secondary alcohol provided in step a) has a boiling point of below 200° C., preferably of below 175° C., more preferably below 145° C., and most preferably below 125° C.

According to one embodiment of the present invention, the alcohol provided in step a) is a primary alcohol.

According to one embodiment of the present invention, the primary or secondary alcohol provided in step a) is a primary or secondary alcohol having a branched or linear C₂-C₁₂-alkyl chain, preferably a branched or linear C₂-C₆-alkyl chain, or the primary or secondary alcohol provided in step a) is the selected from the group consisting of ethanol, n-propanol, n-butanol, n-pentanol, n-hexanol, n-heptanol, n-octanol, 2-propanol, 2-methyl-1-propanol, 2-butanol, 3-methyl-2-butanol, 2-butanol and 3-pentanol.

According to one embodiment of the present invention, the surface-reacted calcium carbonate provided in step b) has a volume median particle size (d₅₀) from 1.0 to 75 μm, preferably from 2 to 50 μm, more preferably 3 to 40 μm, even more preferably from 4 to 30 μm, and most preferably from 5 to 15 μm, and/or a top cut (d₉₈) value from 2 to 150 μm, preferably from 4 to 100 μm, more preferably 6 to 80 μm, even more preferably from 8 to 60 μm, and most preferably from 10 to 30 μm.

According to one embodiment of the present invention, the surface-reacted calcium carbonate provided in step b) has a specific surface area (BET) from 15 to 200 m²/g, preferably of from 27 to 180 m²/g, more preferably from 30 to 180 m²/g, even more preferably 45 to 180 m²/g, and most preferably from 120 to 180 m²/g, as measured by the BET method.

According to one embodiment of the present invention, the surface-reacted calcium carbonate provided in step b) has intra-particle intruded specific pore volume in the range from 0.10 to 2.3 cm³/g, more preferably from 0.20 to 2.0 cm³/g, even more preferably from 0.40 to 1.8 cm³/g and most preferably from 0.70 to 1.6 cm³/g, calculated from mercury porosimetry measurement.

According to one embodiment of the present invention, the one or more H₃O⁺ ion donors are selected from the group consisting of hydrochloric acid, sulfuric acid, sulfurous acid, phosphoric acid, citric acid, oxalic acid, an acidic salt, acetic acid, formic acid, and mixtures thereof, preferably selected from the group consisting of hydrochloric acid, sulfuric acid, sulfurous acid, phosphoric acid, oxalic acid, H₂PO₄⁻, being at least partially neutralized by a corresponding cation such as Li⁺, Na⁺ or K⁺, HPO₄²⁻, being at least partially neutralized by a corresponding cation such as Li⁺, Na⁺, K⁺, Mg²⁺, or Ca²⁺ and mixtures thereof, more preferably selected from the group consisting of hydrochloric acid, sulfuric acid, sulfurous acid, phosphoric acid, oxalic acid, or mixtures thereof, and most preferably the one or more H₃O⁺ ion donor is phosphoric acid.

According to one embodiment of the present invention, the surface-reacted calcium carbonate is a reaction product of ground natural calcium carbonate (GNCC) with carbon dioxide and one or more H₃O⁺ ion donors, wherein the one or more H₃O⁺ ion donor is phosphoric acid, and wherein the carbon dioxide is formed in situ by the H₃O⁺ ion donors treatment.

According to one embodiment of the present invention, the surface-reacted calcium carbonate provided in step b) has a total number of basic sites from 0.01 to 0.6 mmol/g, preferably from 0.05 to 0.5 mmol/g, more preferably from 0.10 to 0.45 mmol/g, based on the total dry weight of the surface-reacted calcium carbonate, determined by temperature-programmed desorption with carbon dioxide, and/or a total number of acidic sites from 0.01 to 0.6 mmol/g, preferably from 0.05 to 0.5 mmol/g, more preferably from 0.10 to 0.45 mmol/g, based on the total dry weight of the surface-reacted calcium carbonate, determined by temperature-programmed desorption with ammonia.

According to one embodiment of the present invention, the surface-reacted calcium carbonate is dried prior to step d) at a temperature in the range of from 100 to 500° C., preferably from 300 to 475° C., and/or the surface-reacted calcium carbonate used in step d) has a residual total moisture content from 0.01 wt.-% to 0.75 wt.-%, preferably from 0.02 wt.-% to 0.5 wt.-%, based on the total dry weight of the surface-reacted calcium carbonate.

According to one embodiment of the present invention, the vaporized alcohol obtained in step c) is present in a gaseous feed stream in an amount of at least 2 vol. %, preferably in a range of 10 to 80 vol. %, more preferably 10 to 55 vol. %, and most preferably in the range of 10 to 40 vol. %, and/or the vaporized alcohol obtained in step c) is mixed with a carrier gas selected from the group consisting of helium, nitrogen, argon, and mixtures thereof, preferably nitrogen.

According to one embodiment of the present invention, step d) is carried out at a reaction temperature in the range of from 150 to 500° C., preferably from 150 to 475° C., more preferably from 250 to 475° C., and most preferably from above 375 to 475° C.

According to one embodiment of the present invention, the surface-reacted calcium carbonate is used in a Guerbet self-condensation reaction of a primary or secondary alcohol as defined herein.

For the present invention, the following terms have the following meanings:

A “β-hydrogen” is a hydrogen atom attached to a carbon atom in β-position of the hydroxy group (—OH) of the alcohol provided in step a) of the process according to the invention (or the alcohol provided for the use according to the invention). A carbon atom in β-position to a hydroxy group refers to a carbon atom that is directly attached to the carbon atom (α-position) being attached to the hydroxy group. For ethanol, the β-position can be illustrated as follows:

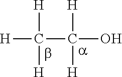

A “Guerbet self-condensation reaction” is a condensation reaction of two of the same alcohol molecules (e.g. two molecules of the same primary or secondary alcohol) with the release of one water molecule resulting in an alcohol product containing the sum of the carbon atoms of the two alcohol molecules. For example, the Guerbet self-condensation reaction of ethanol (C2-alcohol) yields n-butanol (C4-alcohol).

Guerbet self-condensation reactions of alcohols are well-known in the art. It is currently believed in the art that the reaction proceeds stepwise in that two molecules of the same primary or secondary alcohol substrates first each undergo a dehydrogenation to provide two aldehydes or ketones, respectively, which then undergo an aldol condensation to form an alpha, beta-unsaturated aldehyde or ketone. The alpha, beta-unsaturated aldehyde or ketone is then fully hydrogenated to give the final alcohol product. The current understanding in the art of the reaction mechanism of the Guerbet self-condensation is only included herein for illustrative purposes, and should not be construed as limiting the process according to the invention in any way.

As will be readily understood by those skilled in the art, a “primary or secondary alcohol having at least one β-hydrogen” as defined herein may be dehydrogenated to an aldehyde or a ketone that is enolizable. The enolizable aldehyde or ketone may then react in an aldol condensation.

A “gas phase reaction” or a “reaction in the gas phase” is to be understood as a chemical reaction comprising a gaseous reactant, e.g. the primary or secondary alcohol as defined herein, which is optionally diluted in a non-reactive carrier gas (e.g. nitrogen).

A “surface-reacted calcium carbonate” according to the present invention is a reaction product of natural ground calcium carbonate (GNCC) or precipitated calcium carbonate (PCC) treated with carbon dioxide and one or more H₃O⁺ ion donors, wherein the carbon dioxide is formed in situ by the H₃O⁺ ion donors treatment. An H₃O⁺ ion donor in the context of the present invention is a Bronsted acid and/or an acid salt. The terms “natural ground calcium carbonate” and “ground natural calcium carbonate” are used interchangeably herein and refer to the same material.

Throughout the present document, the term “specific surface area” (“SSA”, in m²/g), which is used to define functionalized calcium carbonate or other materials, refers to the specific surface area as determined by using the BET method (using nitrogen as adsorbing gas), according to ISO 9277:2010.

The “particle size” of surface-reacted calcium carbonate herein is described as volume-based particle size distribution dₓ (vol). Therein, the value dₓ (vol) represents the diameter relative to which x % by volume of the particles have diameters less than dₓ (vol). This means that, for example, the d₂₀ (vol) value is the particle size at which 20 vol. % of all particles are smaller than that particle size. The d₅₀ (vol) value is thus the volume median particle size, i.e. 50 vol. % of all particles are smaller than that particle size and the d₉₈ (vol) value, referred to as volume-based top cut, is the particle size at which 98 vol. % of all particles are smaller than that particle size. The volume-based particle size distribution dₓ (vol) can be determined by laser diffraction.

Volume median particle size d₅₀ was evaluated using a Malvern Mastersizer 3000 Laser Diffraction System. The d₅₀ or d₉₈ value, measured using a Malvern Mastersizer 3000 Laser Diffraction System, indicates a diameter value such that 50% or 98% by volume, respectively, of the particles have a diameter of less than this value. The raw data obtained by the measurement are analysed using the Mie theory, with a particle refractive index of 1.57 and an absorption index of 0.005.

For the purpose of the present invention, the “porosity” or “pore volume” refers to the intra-particle intruded specific pore volume.

In the context of the present invention, the term “pore” is to be understood as describing the space that is found between and/or within particles, i.e. that is formed by the particles as they pack together under nearest neighbor contact (interparticle pores), such as in a powder or a compact, and/or the void space within porous particles (intraparticle pores), and that allows the passage of liquids under pressure when saturated by the liquid and/or supports absorption of surface wetting liquids.

The specific pore volume is measured using a mercury intrusion porosimetry measurement using a Micromeritics Autopore V 9620 mercury porosimeter having a maximum applied pressure of mercury 414 MPa (60 000 psi), equivalent to a Laplace throat diameter of 0.004 μm. The equilibration time used at each pressure step is 20 s. The sample material is sealed in a 3 cm³ chamber powder penetrometer for analysis. The data are corrected for mercury compression, penetrometer expansion and sample material elastic compression using the software Pore-Comp (Gane, P. A. C., Kettle, J. P., Matthews, G. P. and Ridgway, C. J., “Void Space Structure of Compressible Polymer Spheres and Consolidated Calcium Carbonate Paper-Coating Formulations”, Industrial and Engineering Chemistry Research, 1996, 35(5), 1753-1764).

The total pore volume seen in the cumulative intrusion data is separated into two regions with the intrusion data from 214 μm down to about 1 to 4 μm showing the coarse packing of the sample between any agglomerate structures contributing strongly. Below these diameters lies the fine interparticle packing of the particles themselves. If they also have intraparticle pores, then this region appears bimodal, and by taking the specific pore volume intruded by mercury into pores finer than the modal turning point, i.e. finer than the bimodal point of inflection, we thus define the specific intraparticle pore volume. The sum of these three regions gives the total overall pore volume of the powder, but depends strongly on the original sample compaction/settling of the powder at the coarse pore end of the distribution.

By taking the first derivative of the cumulative intrusion curve, the pore size distributions based on equivalent Laplace diameter, inevitably including pore-shielding, are revealed. The differential curves clearly show the coarse agglomerate pore structure region, the interparticle pore region and the intraparticle pore region, if present. Knowing the intraparticle pore diameter range, it is possible to subtract the remainder interparticle and interagglomerate pore volume from the total pore volume to deliver the desired pore volume of the internal pores alone in terms of the pore volume per unit mass (specific pore volume). The same principle of subtraction, of course, applies for isolating any of the other pore size regions of interest.

A “dry” material (e.g., dry surface-reacted calcium carbonate) in the meaning of the present invention has a total or residual moisture content which, unless specified otherwise, is less than or equal to 5.0 wt. %, preferably less than or equal to 0.75 wt. %, more preferably less than or equal to 0.5 wt. %, even more preferably less than or equal to 0.2 wt. %, and most preferably between 0.02 and 0.07 wt. %, based on the total weight of the dried material.

The “total number of basic sites” is a measure of the basicity of a solid material and is represented by the total molar amount of carbon dioxide that can be adsorbed on the basic sites of a certain amount of the solid material, and is determined by temperature-programmed desorption with carbon dioxide as described herein.

The “total number of acidic sites” is a measure of the acidity of a solid material and is represented by the total molar amount of ammonia that can be adsorbed on the acidic sites of a certain amount of the solid material, and is determined by temperature-programmed desorption with ammonia as described herein.

Where the term “comprising” is used in the present description and claims, it does not exclude other non-specified elements of major or minor functional importance. For the purposes of the present invention, the term “essentially consisting of” and “consisting of” are considered to be specific embodiments of the term “comprising of”. If hereinafter a group is defined to comprise at least a certain number of embodiments, this is also to be understood to disclose a group, which optionally essentially consists only of these embodiments or consists only of these embodiments.

Whenever the terms “including” or “having” are used, these terms are meant to be equivalent to “comprising” as defined above.

Where an indefinite or definite article is used when referring to a singular noun, e.g. “a”, “an” or “the”, this includes a plural of that noun unless something else is specifically stated.

Terms like “obtainable” or “definable” and “obtained” or “defined” are used interchangeably. This e.g. means that, unless the context clearly dictates otherwise, the term “obtained” does not mean to indicate that, e.g., an embodiment must be obtained by, e.g., the sequence of steps following the term “obtained” even though such a limited understanding is always included by the terms “obtained” or “defined” as a preferred embodiment.

### DETAILED DESCRIPTION OF THE INVENTION

### The Process According to the Invention

One aspect of the present invention relates to a process for preparing an alcohol by a Guerbet self-condensation reaction in the gas phase. The process comprises the steps of:
a) providing a primary or secondary alcohol, wherein the primary or secondary alcohol has at least one β-hydrogen;
b) providing a surface-reacted calcium carbonate, wherein the surface-reacted calcium carbonate is a reaction product of ground natural calcium carbonate (GNCC) or precipitated calcium carbonate (PCC) with carbon dioxide and one or more H₃O⁺ ion donors and wherein the carbon dioxide is formed in situ by the H₃O⁺ ion donors treatment and/or is supplied from an external source, and wherein the surface-reacted calcium carbonate has a specific surface area of at least 15 m²/g, measured using nitrogen and the BET method according to ISO 9277:2010;
c) vaporizing the alcohol provided in step a);
d) reacting the vaporized alcohol obtained in step c) in the presence of the surface-reacted calcium carbonate provided in step b) as a catalyst.

### Step a)

The process includes a step a) of providing a primary or secondary alcohol, wherein the primary or secondary alcohol has at least one β-hydrogen.

The primary or secondary alcohol has at least one β-hydrogen. As will be understood by the person skilled in the art, the overall number of β-hydrogens depends on the structure of the alcohol provided in step a). If a primary alcohol is provided in step a), the alcohol can have from one to three β-hydrogens depending on the substitution pattern on the β-carbon atom of the alcohol. For example, ethanol has three β-hydrogens, n-propanol has two β-hydrogens, and 2-methyl-1-propanol has one β-hydrogen. In a preferred embodiment, the alcohol is a primary alcohol having two or three β-hydrogens.

If a secondary alcohol is provided in step a), the alcohol has two β-carbon atoms and therefore can have from one to six β-hydrogens (depending on the substituents on the β-carbon atoms). In a preferred embodiment, the alcohol is a secondary alcohol having at least one β-carbon atom, which contains two or three β-hydrogens.

According to one preferred embodiment, the primary or secondary alcohol provided in step a) is an alcohol which essentially does not decompose (or does not decompose) upon vaporization.

The primary or secondary alcohol can have a defined boiling point. For example, the alcohol may have a boiling point of below 300° C., e.g. in the range of 60 to 300° C. It is preferred that the boiling point of the alcohol is below 200° C., preferably below 175° C., more preferably below 145° C., and most preferably below 125° C. It is particularly preferred that the boiling point of the primary or secondary alcohol is in the range of from 60 to 145° C. or in the range of from 60 to 125° C.

According to one preferred embodiment, the alcohol provided in step a) is a primary alcohol having a boiling point of below 200° C. (e.g. in the range of 60 to 200° C.), preferably below 175° C., more preferably below 145° C., and most preferably below 125° C.

The alcohol can be a primary or a secondary alcohol. In one embodiment, the alcohol is a secondary alcohol. In another embodiment, the alcohol is a primary alcohol. It is preferred that the alcohol is a primary alcohol.

The primary or secondary alcohol can be an alcohol having a branched alkyl chain (e.g. 2-methyl-1-propanol, 3-methyl-2-butanol), a linear alkyl chain (e.g. ethanol, n-proponal, n-butanol, n-pentanol, 2-propanol, 2-butanol) or a cyclic alkyl chain (e.g. cyclopentanol). The primary or secondary alcohol can be an alcohol having a branched alkyl chain (e.g. 2-methyl-1-propanol, 3-methyl-2-butanol), a linear alkyl chain (e.g. ethanol, n-propanol, n-butanol, n-pentanol, 2-propanol, 2-butanol) or a cyclic alkyl chain (e.g. cyclopentanol).

Preferably, the primary or secondary alcohol is an alcohol having a branched C₂-C₁₂-alkyl chain, more preferably a branched C₂-C₆-alkyl chain, or a linear C₂-C₁₂-alkyl chain, more preferably a linear C₂-C₆-alkyl chain.

In a preferred embodiment, the primary or secondary alcohol has a linear alkyl chain. In another preferred embodiment, the alcohol provided in step a) is a primary alcohol having a linear alkyl chain, preferably a linear C₂-C₁₂-alkyl chain, more preferably a C₂-C₆-alkyl chain.

According to one preferred embodiment, the alcohol provided in step a) is selected from the group consisting of ethanol, n-propanol, n-butanol, n-pentanol, n-hexanol, n-heptanol, n-octanol, 2-propanol, 2-methyl-1-propanol, 2-butanol, 3-methyl-2-butanol, 2-butanol and 3-pentanol, preferably from the group consisting of ethanol, n-propanol, n-butanol, 2-propanol, 2-butanol and 2-pentanol.

In one specific embodiment, the alcohol provided in step a) is ethanol.

### Step b)

In step b) of the process according to the invention, a surface-reacted calcium carbonate is provided. The surface-reacted calcium carbonate is a reaction product of ground natural calcium carbonate (GNCC) or precipitated calcium carbonate (PCC) with carbon dioxide and one or more H₃O⁺ ion donors and wherein the carbon dioxide is formed in situ by the H₃O⁺ ion donors treatment and/or is supplied from an external source. The surface-reacted calcium carbonate has a specific surface area of at least 15 m²/g, measured using nitrogen and the BET method according to ISO 9277:2010.

In a preferred embodiment of the invention, the surface-reacted calcium carbonate is obtained by a process comprising the steps of: (a) providing a suspension of natural or precipitated calcium carbonate, (b) adding at least one acid having a pKₐ value of 0 or less at 20° C. or having a pKₐ value from 0 to 2.5 at 20° C. to the suspension of step (a), and (c) treating the suspension of step (a) with carbon dioxide before, during or after step (b). According to another embodiment the surface-reacted calcium carbonate is obtained by a process comprising the steps of: (A) providing a natural or precipitated calcium carbonate, (B) providing at least one water-soluble acid, (C) providing gaseous CO₂, (D) contacting said natural or precipitated calcium carbonate of step (A) with the at least one acid of step (B) and with the CO₂ of step (C), characterized in that: (i) the at least one acid of step B) has a pKₐ of greater than 2.5 and less than or equal to 7 at 20° C., associated with the ionization of its first available hydrogen, and a corresponding anion is formed on loss of this first available hydrogen capable of forming a water-soluble calcium salt, and (ii) following contacting the at least one acid with natural or precipitated calcium carbonate, at least one water-soluble salt, which in the case of a hydrogen-containing salt has a pKₐ of greater than 7 at 20° C., associated with the ionization of the first available hydrogen, and the salt anion of which is capable of forming water-insoluble calcium salts, is additionally provided.

“Natural ground calcium carbonate” (GNCC) preferably is selected from calcium carbonate containing minerals selected from the group comprising marble, chalk, limestone and mixtures thereof. Natural calcium carbonate may comprise further naturally occurring components such as alumino silicate etc.

In general, the grinding of natural ground calcium carbonate may be a dry or wet grinding step and may be carried out with any conventional grinding device, for example, under conditions such that comminution predominantly results from impacts with a secondary body, i.e. in one or more of: a ball mill, a rod mill, a vibrating mill, a roll crusher, a centrifugal impact mill, a vertical bead mill, an attrition mill, a pin mill, a hammer mill, a pulverizer, a shredder, a de-clumper, a knife cutter, or other such equipment known to the skilled man. In case the calcium carbonate containing mineral material comprises a wet ground calcium carbonate containing mineral material, the grinding step may be performed under conditions such that autogenous grinding takes place and/or by horizontal ball milling, and/or other such processes known to the skilled man. The wet processed ground calcium carbonate containing mineral material thus obtained may be washed and dewatered by well-known processes, e.g. by flocculation, filtration or forced evaporation prior to drying. The subsequent step of drying (if necessary) may be carried out in a single step such as spray drying, or in at least two steps. It is also common that such a mineral material undergoes a beneficiation step (such as a flotation, bleaching or magnetic separation step) to remove impurities.

“Precipitated calcium carbonate” (PCC) in the meaning of the present invention is a synthesized material, generally obtained by precipitation following reaction of carbon dioxide and calcium hydroxide in an aqueous environment or by precipitation of calcium and carbonate ions, for example CaCl₂ and Na₂CO₃, out of solution. Further possible ways of producing PCC are the lime soda process, or the Solvay process in which PCC is a by-product of ammonia production. Precipitated calcium carbonate exists in three primary crystalline forms: calcite, aragonite and vaterite, and there are many different polymorphs (crystal habits) for each of these crystalline forms. Calcite has a trigonal structure with typical crystal habits such as scalenohedral (S-PCC), rhombohedral (R-PCC), hexagonal prismatic, pinacoidal, colloidal (C-PCC), cubic, and prismatic (P-PCC). Aragonite is an orthorhombic structure with typical crystal habits of twinned hexagonal prismatic crystals, as well as a diverse assortment of thin elongated prismatic, curved bladed, steep pyramidal, chisel shaped crystals, branching tree, and coral or worm-like form. Vaterite belongs to the hexagonal crystal system. The obtained PCC slurry can be mechanically dewatered and dried.

According to one embodiment of the present invention, the precipitated calcium carbonate is precipitated calcium carbonate, preferably comprising aragonitic, vateritic or calcitic mineralogical crystal forms or mixtures thereof.

Precipitated calcium carbonate may be ground prior to the treatment with carbon dioxide and at least one H₃O⁺ ion donor by the same means as used for grinding natural calcium carbonate as described above.

According to one embodiment of the present invention, the natural or precipitated calcium carbonate is in form of particles having a weight median particle size d₅₀ of 0.05 to 10.0 μm, preferably 0.2 to 5.0 μm, more preferably 0.4 to 3.0 μm, most preferably 0.6 to 1.2 μm, especially 0.7 μm. According to a further embodiment of the present invention, the natural or precipitated calcium carbonate is in form of particles having a top cut particle size des of 0.15 to 55 μm, preferably 1 to 40 μm, more preferably 2 to 25 μm, most preferably 3 to 15 μm, especially 4 μm.

The natural and/or precipitated calcium carbonate may be used dry or suspended in water. Preferably, a corresponding slurry has a content of natural or precipitated calcium carbonate within the range of 1 wt.-% to 90 wt.-%, more preferably 3 wt.-% to 60 wt.-%, even more preferably 5 wt.-% to 40 wt.-%, and most preferably 10 wt.-% to 25 wt.-% based on the weight of the slurry.

The one or more H₃O⁺ ion donor used for the preparation of surface reacted calcium carbonate may be any strong acid, medium-strong acid, or weak acid, or mixtures thereof, generating H₃O⁺ ions under the preparation conditions. According to the present invention, the at least one H₃O⁺ ion donor can also be an acidic salt, generating H₃O⁺ ions under the preparation conditions.

According to one embodiment, the at least one H₃O⁺ ion donor is a strong acid having a pKₐ of 0 or less at 20° C.

According to another embodiment, the at least one H₃O⁺ ion donor is a medium-strong acid having a pKₐ value from 0 to 2.5 at 20° C. If the pKₐ at 20° C. is 0 or less, the acid is preferably selected from sulfuric acid, hydrochloric acid, or mixtures thereof. If the pKₐ at 20° C. is from 0 to 2.5, the H₃O⁺ ion donor is preferably selected from H₂SO₃, H₃PO₄, oxalic acid, or mixtures thereof. The at least one H₃O⁺ ion donor can also be an acidic salt, for example, HSO₄⁻ or H₂PO₄⁻, being at least partially neutralized by a corresponding cation such as Li⁺, Na⁺ or K⁺, or HPO₄²⁻, being at least partially neutralized by a corresponding cation such as Li⁺, Na⁺, K⁺, Mg²⁺ or Ca²⁺. The at least one H₃O⁺ ion donor can also be a mixture of one or more acids and one or more acidic salts.

According to still another embodiment, the at least one H₃O⁺ ion donor is a weak acid having a pKₐ value of greater than 2.5 and less than or equal to 7, when measured at 20° C., associated with the ionization of the first available hydrogen, and having a corresponding anion, which is capable of forming water-soluble calcium salts. Subsequently, at least one water-soluble salt, which in the case of a hydrogen-containing salt has a pKₐ of greater than 7, when measured at 20° C., associated with the ionization of the first available hydrogen, and the salt anion of which is capable of forming water-insoluble calcium salts, is additionally provided. According to the preferred embodiment, the weak acid has a pKₐ value from greater than 2.5 to 5 at 20° C., and more preferably the weak acid is selected from the group consisting of acetic acid, formic acid, propanoic acid, and mixtures thereof. Exemplary cations of said water-soluble salt are selected from the group consisting of potassium, sodium, lithium and mixtures thereof. In a more preferred embodiment, said cation is sodium or potassium. Exemplary anions of said water-soluble salt are selected from the group consisting of phosphate, dihydrogen phosphate, monohydrogen phosphate, oxalate, silicate, mixtures thereof and hydrates thereof. In a more preferred embodiment, said anion is selected from the group consisting of phosphate, dihydrogen phosphate, monohydrogen phosphate, mixtures thereof and hydrates thereof. In a most preferred embodiment, said anion is selected from the group consisting of dihydrogen phosphate, monohydrogen phosphate, mixtures thereof and hydrates thereof. Water-soluble salt addition may be performed dropwise or in one step. In the case of drop wise addition, this addition preferably takes place within a time period of 10 minutes. It is more preferred to add said salt in one step.

According to one embodiment of the present invention, the at least one H₃O⁺ ion donor is selected from the group consisting of hydrochloric acid, sulfuric acid, sulfurous acid, phosphoric acid, citric acid, oxalic acid, acetic acid, formic acid, and mixtures thereof. Preferably the at least one H₃O⁺ ion donor is selected from the group consisting of hydrochloric acid, sulfuric acid, sulfurous acid, phosphoric acid, oxalic acid, H₂PO₄⁻, being at least partially neutralized by a corresponding cation such as Li⁺, Na⁺ or K⁺, HPO₄²⁻, being at least partially neutralized by a corresponding cation such as Li⁺, Na⁺, K⁺, Mg²⁺, or Ca²⁺ and mixtures thereof, more preferably the at least one H₃O⁺ ion donor is selected from the group consisting of hydrochloric acid, sulfuric acid, sulfurous acid, phosphoric acid, oxalic acid, or mixtures thereof, and most preferably, the at least one H₃O⁺ ion donor is phosphoric acid.

The one or more H₃O⁺ ion donor can be added to the suspension as a concentrated solution or a more diluted solution. Preferably, the molar ratio of the H₃O⁺ ion donor to the natural or precipitated calcium carbonate is from 0.01 to 4, more preferably from 0.02 to 2, even more preferably 0.05 to 1 and most preferably 0.1 to 0.58.

As an alternative, it is also possible to add the H₃O⁺ ion donor to the water before the natural or precipitated calcium carbonate is suspended.

In a next step, the natural or precipitated calcium carbonate is treated with carbon dioxide. If a strong acid such as sulfuric acid or hydrochloric acid is used for the H₃O⁺ ion donor treatment of the natural or precipitated calcium carbonate, the carbon dioxide is automatically formed. Alternatively or additionally, the carbon dioxide can be supplied from an external source.

H₃O⁺ ion donor treatment and treatment with carbon dioxide can be carried out simultaneously which is the case when a strong or medium-strong acid is used. It is also possible to carry out H₃O⁺ ion donor treatment first, e.g. with a medium strong acid having a pKₐ in the range of 0 to 2.5 at 20° C., wherein carbon dioxide is formed in situ, and thus, the carbon dioxide treatment will automatically be carried out simultaneously with the H₃O⁺ ion donor treatment, followed by the additional treatment with carbon dioxide supplied from an external source.

In a preferred embodiment, the H₃O⁺ ion donor treatment step and/or the carbon dioxide treatment step are repeated at least once, more preferably several times. According to one embodiment, the at least one H₃O⁺ ion donor is added over a time period of at least about 5 min, preferably at least about 10 min, typically from about 10 to about 20 min, more preferably about 30 min, even more preferably about 45 min, and sometimes about 1 h or more.

Subsequent to the H₃O⁺ ion donor treatment and carbon dioxide treatment, the pH of the aqueous suspension, measured at 20° C., naturally reaches a value of greater than 6.0, preferably greater than 6.5, more preferably greater than 7.0, even more preferably greater than 7.5, thereby preparing the surface-reacted natural or precipitated calcium carbonate as an aqueous suspension having a pH of greater than 6.0, preferably greater than 6.5, more preferably greater than 7.0, even more preferably greater than 7.5.

In a particular preferred embodiment the surface reacted calcium carbonate is a reaction product of natural ground calcium carbonate (GNCC) with carbon dioxide and phosphoric acid, wherein the carbon dioxide is formed in situ by the phosphoric acid treatment.

Further details about the preparation of the surface-reacted natural calcium carbonate are disclosed in WO 00/39222 A1, WO 2004/083316 A1, WO 2005/121257 A2, WO 2009/074492 A1, EP 2 264 108 A1, EP 2 264 109 A1 and US 2004/0020410 A1, the content of these references herewith being included in the present application.

Similarly, surface-reacted precipitated calcium carbonate is obtained. As can be taken in detail from WO 2009/074492 A1, surface-reacted precipitated calcium carbonate is obtained by contacting precipitated calcium carbonate with H₃O⁺ ions and with anions being solubilized in an aqueous medium and being capable of forming water-insoluble calcium salts, in an aqueous medium to form a slurry of surface-reacted precipitated calcium carbonate, wherein said surface-reacted precipitated calcium carbonate comprises an insoluble, at least partially crystalline calcium salt of said anion formed on the surface of at least part of the precipitated calcium carbonate.

Said solubilized calcium ions correspond to an excess of solubilized calcium ions relative to the solubilized calcium ions naturally generated on dissolution of precipitated calcium carbonate by H₃O⁺ ions, where said H₃O⁺ ions are provided solely in the form of a counterion to the anion, i.e. via the addition of the anion in the form of an acid or non-calcium acid salt, and in absence of any further calcium ion or calcium ion generating source.

Said excess solubilized calcium ions are preferably provided by the addition of a soluble neutral or acid calcium salt, or by the addition of an acid or a neutral or acid non-calcium salt which generates a soluble neutral or acid calcium salt in situ.

Said H₃O⁺ ions may be provided by the addition of an acid or an acid salt of said anion, or the addition of an acid or an acid salt which simultaneously serves to provide all or part of said excess solubilized calcium ions.

In a further preferred embodiment of the preparation of the surface-reacted natural or precipitated calcium carbonate, the natural or precipitated calcium carbonate is reacted with the one or more H₃O⁺ ion donors and/or the carbon dioxide in the presence of at least one compound selected from the group consisting of silicate, silica, aluminium hydroxide, earth alkali aluminate such as sodium or potassium aluminate, magnesium oxide, or mixtures thereof. Preferably, the at least one silicate is selected from an aluminium silicate, a calcium silicate, or an earth alkali metal silicate. These components can be added to an aqueous suspension comprising the natural or precipitated calcium carbonate before adding the one or more H₃O⁺ ion donors and/or carbon dioxide.

Alternatively, the silicate and/or silica and/or aluminium hydroxide and/or earth alkali aluminate and/or magnesium oxide component(s) can be added to the aqueous suspension of natural or precipitated calcium carbonate while the reaction of natural or precipitated calcium carbonate with the one or more H₃O⁺ ion donors and carbon dioxide has already started. Further details about the preparation of the surface-reacted natural or precipitated calcium carbonate in the presence of at least one silicate and/or silica and/or aluminium hydroxide and/or earth alkali aluminate component(s) are disclosed in WO 2004/083316 A1, the content of this reference herewith being included in the present application.

The surface-reacted calcium carbonate can be kept in suspension, optionally further stabilized by a dispersant. Conventional dispersants known to the skilled person can be used. A preferred dispersant is comprised of polyacrylic acids and/or carboxymethylcelluloses.

Alternatively, the aqueous suspension described above can be dried, thereby obtaining the solid (i.e. dry or containing as little water that it is not in a fluid form) surface-reacted natural or precipitated calcium carbonate in the form of granules or a powder.

In a particularly preferred embodiment of the present invention, the surface-reacted calcium carbonate is a reaction product of natural ground calcium carbonate with carbon dioxide and one or more H₃O⁺ ion donors, wherein the carbon dioxide is formed in situ by the H₃O⁺ ion donors treatment, and wherein the one or more H₃O⁺ ion donor is phosphoric acid. In said embodiment, it is preferred that the surface-reacted calcium carbonate comprises phosphate groups and has an atomic ratio of calcium to phosphorus atoms of at most 3.0, more preferably at most 2.5, and most preferably at most 2.3, determined by XPS.

It is to be understood that the surface-reacted calcium carbonate is not a calcined material.

In a preferred embodiment, the surface-reacted calcium carbonate has a specific surface area of from 15 m²/g to 200 m²/g, preferably from 27 m²/g to 180 m²/g, more preferably from 30 m²/g to 180 m²/g, even more preferably from 45 m²/g to 180 m²/g, most preferably from 120 m²/g to 180 m²/g, measured using nitrogen and the BET method. For example, the surface-reacted calcium carbonate has a specific surface area of from 150 m²/g to 180 m²/g, measured using nitrogen and the BET method. The BET specific surface area in the meaning of the present invention is defined as the surface area of the particles divided by the mass of the particles. As used therein the specific surface area is measured by adsorption using the BET isotherm (ISO 9277:2010) and is specified in m²/g.

It is furthermore preferred that the surface-reacted calcium carbonate particles have a volume median particle size d₅₀ (vol) of from 1.0 to 75 μm, preferably from 2 to 50 μm, more preferably 3 to 40 μm, even more preferably from 4 to 30 μm, and most preferably from 5 to 15 μm.

It may furthermore be preferred that the surface-reacted calcium carbonate particles have a top cut des (vol) value of from 2 to 150 μm, preferably from 4 to 100 μm, more preferably 6 to 80 μm, even more preferably from 8 to 60 μm, and most preferably from 10 to 30 μm.

The value dₓ represents the diameter relative to which x % of the particles have diameters less than dₓ. This means that the des value is the particle size at which 98% of all particles are smaller. The des value is also designated as “top cut”. The dₓ values may be given in volume or weight percent. The d₅₀ (wt) value is thus the weight median particle size, i.e. 50 wt.-% of all grains are smaller than this particle size, and the d₅₀ (vol) value is the volume median particle size, i.e. 50 vol.-% of all grains are smaller than this particle size.

Volume median grain diameter d₅₀ was evaluated using a Malvern Mastersizer 3000 Laser Diffraction System. The d₅₀ or des value, measured using a Malvern Mastersizer 3000 Laser Diffraction System, indicates a diameter value such that 50% or 98% by volume, respectively, of the particles have a diameter of less than this value. The raw data obtained by the measurement are analysed using the Mie theory, with a particle refractive index of 1.57 and an absorption index of 0.005.

The weight median grain diameter of the natural ground calcium carbonate and precipitated calcium carbonate is determined by the sedimentation method, which is an analysis of sedimentation behaviour in a gravimetric field. The measurement is made with a Sedigraph™ 5120, Micromeritics Instrument Corporation. The method and the instrument are known to the skilled person and are commonly used to determine grain size of fillers and pigments. The measurement is carried out in an aqueous solution of 0.1 wt.-% Na₄P₂O₇. The samples were dispersed using a high-speed stirrer and sonicated.

The processes and instruments are known to the skilled person and are commonly used to determine grain size of fillers and pigments.

The specific pore volume is measured using a mercury intrusion porosimetry measurement using a Micromeritics Autopore V 9620 mercury porosimeter having a maximum applied pressure of mercury 414 MPa (60 000 psi), equivalent to a Laplace throat diameter of 0.004 μm (˜nm). The equilibration time used at each pressure step is 20 seconds. The sample material is sealed in a 5 cm³ chamber powder penetrometer for analysis. The data are corrected for mercury compression, penetrometer expansion and sample material compression using the software Pore-Comp (Gane, P. A. C., Kettle, J. P., Matthews, G. P. and Ridgway, C. J., “Void Space Structure of Compressible Polymer Spheres and Consolidated Calcium Carbonate Paper-Coating Formulations”, Industrial and Engineering Chemistry Research, 35(5), 1996, p 1753-1764.).

The total pore volume seen in the cumulative intrusion data can be separated into two regions with the intrusion data from 214 μm down to about 1-4 μm showing the coarse packing of the sample between any agglomerate structures contributing strongly. Below these diameters lies the fine interparticle packing of the particles themselves. If they also have intraparticle pores, then this region appears bi modal, and by taking the specific pore volume intruded by mercury into pores finer than the modal turning point, i.e. finer than the bi-modal point of inflection, the specific intraparticle pore volume is defined. The sum of these three regions gives the total overall pore volume of the powder, but depends strongly on the original sample compaction/settling of the powder at the coarse pore end of the distribution.

By taking the first derivative of the cumulative intrusion curve the pore size distributions based on equivalent Laplace diameter, inevitably including pore-shielding, are revealed. The differential curves clearly show the coarse agglomerate pore structure region, the interparticle pore region and the intraparticle pore region, if present. Knowing the intraparticle pore diameter range it is possible to subtract the remainder interparticle and interagglomerate pore volume from the total pore volume to deliver the desired pore volume of the internal pores alone in terms of the pore volume per unit mass (specific pore volume). The same principle of subtraction, of course, applies for isolating any of the other pore size regions of interest.

Preferably, the surface-reacted calcium carbonate has an intra-particle intruded specific pore volume in the range from 0.1 to 2.3 cm³/g, more preferably from 0.2 to 2.0 cm³/g, especially preferably from 0.4 to 1.8 cm³/g and most preferably from 0.6 to 1.6 cm³/g, calculated from mercury porosimetry measurement.

The intra-particle pore size of the surface-reacted calcium carbonate preferably is in a range of from 0.004 to 1.6 μm, more preferably in a range of from 0.005 to 1.3 μm, especially preferably from 0.006 to 1.15 μm and most preferably of 0.007 to 1.0 μm, determined by mercury porosimetry measurement.

According to one preferred embodiment, the surface-reacted calcium carbonate provided in step b) has a specific surface area (BET) from 10 to 200 m²/g, preferably of from 60 to 200 m²/g, more preferably from 100 to 200 m²/g, even more preferably from 120 to 180 m²/g, and most preferably from 140 to 180 m²/g, as measured by the BET method, and/or an intra-particle intruded specific pore volume in the range from 0.10 to 2.0 cm³/g, more preferably from 0.20 to 2.0 cm³/g, even more preferably from 0.50 to 2.0 cm³/g and most preferably from 0.70 to 1.6 cm³/g, calculated from mercury porosimetry measurement.

According to one preferred embodiment, the surface-reacted calcium carbonate provided in step b) has a specific surface area (BET) from 10 to 200 m²/g, preferably of from 60 to 200 m²/g, more preferably from 100 to 200 m²/g, even more preferably from 120 to 180 m²/g, and most preferably from 140 to 180 m²/g, as measured by the BET method, and an intra-particle intruded specific pore volume in the range from 0.10 to 2.0 cm³/g, more preferably from 0.20 to 2.0 cm³/g, even more preferably from 0.50 to 2.0 cm³/g and most preferably from 0.70 to 1.6 cm³/g, calculated from mercury porosimetry measurement.

According to one preferred embodiment, the surface-reacted calcium carbonate provided in step b) has a volume median particle size (d₅₀) from 0.5 to 50 μm, preferably from 1 to 30 μm, more preferably from 1.5 to 20 μm, and most preferably from 5 to 10 μm, and/or a top cut (d₉₈) value from 1 to 120 μm, preferably from 2 to 100 μm, more preferably from 5 to 50 μm, and most preferably from 12 to 20 μm.

According to one preferred embodiment, the surface-reacted calcium carbonate provided in step b) has a volume median particle size (d₅₀) from 0.5 to 50 μm, preferably from 1 to 30 μm, more preferably from 1.5 to 20 μm, and most preferably from 5 to 10 μm, and a top cut (d₉₈) value from 1 to 120 μm, preferably from 2 to 100 μm, more preferably from 5 to 50 μm, and most preferably from 12 to 20 μm.

According to one preferred embodiment, the surface-reacted calcium carbonate provided in step b) has a total number of basic sites from 0.01 to 0.6 mmol/g, preferably from 0.05 to 0.5 mmol/g, more preferably from 0.10 to 0.45 mmol/g, based on the total dry weight of the surface-reacted calcium carbonate, determined by temperature-programmed desorption with carbon dioxide, and/or
a total number of acidic sites from 0.01 to 0.6 mmol/g, preferably from 0.05 to 0.5 mmol/g, more preferably from 0.10 to 0.45 mmol/g, based on the total dry weight of the surface-reacted calcium carbonate, determined by temperature-programmed desorption with ammonia.

According to one preferred embodiment, the surface-reacted calcium carbonate provided in step b) has a total number of basic sites from 0.01 to 0.6 mmol/g, preferably from 0.05 to 0.5 mmol/g, more preferably from 0.10 to 0.45 mmol/g, based on the total dry weight of the surface-reacted calcium carbonate, determined by temperature-programmed desorption with carbon dioxide, and
a total number of acidic sites from 0.01 to 0.6 mmol/g, preferably from 0.05 to 0.5 mmol/g, more preferably from 0.10 to 0.45 mmol/g, based on the total dry weight of the surface-reacted calcium carbonate, determined by temperature-programmed desorption with ammonia.

According to one preferred embodiment, the surface-reacted calcium carbonate provided in step c) has a total number of basic sites, determined by temperature-programmed desorption with carbon dioxide, relative to a total number of acidic sites, determined by temperature-programmed desorption with ammonia, in the range of 45:55 to 75:25, preferably in the range of from 55:45 to 70:30.

According to one embodiment, the surface-reacted calcium carbonate provided in step b) comprises calcium carbonate and hydroxyapatite. According to one preferred embodiment, the surface-reacted calcium carbonate provided in step b) contains an amount of hydroxylapatite relative to an amount of calcium carbonate in the range of from 10:90 to 90:10, preferably 35:65 to 90:10, more preferably 60:40 to 90:10 (e.g. 70:30 to 85:15), as determined by XRD using the Rietveld method.

### Step c)

In step c) of the process according to the invention, the alcohol provided in step a) is vaporized.

After vaporization, the vaporized alcohol is carried in the gas phase as part of a gaseous feed stream to the surface-reacted calcium carbonate as catalyst for the Guerbet self-condensation reaction (cf. step d).

Suitable heating or vaporizing devices, mass flow controllers, check valves, temperature controller, feed lines for the alcohol and for the carrier gas, alcohol reservoirs, gas bombs, pumps, dispensing lines, reactors, etc can be selected by the skilled person according to the technical requirements and scale of the process.

The alcohol provided in step a) may be fed in liquid form by means of a pump via a feed line to a vaporization reactor where the alcohol is vaporized in accordance with step c). A carrier gas feed line may be connected to the vaporization reactor, and the vaporized alcohol may be admixed with carrier gas to create a gaseous feed stream. The gaseous feed stream comprising the vaporized alcohol may then be fed to the catalyst for reaction.

According to one embodiment, the vaporized alcohol is mixed with a carrier gas to create a gaseous feed stream.

A suitable carrier gas may be helium, nitrogen, argon, hydrogen, or a mixture thereof. Quality, source and/or purity of the carrier gas can be selected by the skilled person. In one specific embodiment, nitrogen is used as carrier gas.

According to one embodiment of the present invention, the vaporized alcohol obtained in step c) is mixed with an inert carrier gas. According to one embodiment of the present invention, the vaporized alcohol obtained in step c) is mixed with a carrier gas selected from the group consisting of helium, nitrogen, argon, hydrogen and mixtures thereof, preferably the carrier gas is nitrogen.

The amount of vaporized alcohol by volume percent of the gaseous feed stream can be adjusted. According to one embodiment, the vaporized alcohol obtained in step c) is present in the gaseous feed stream in an amount of at least 2 vol. %, preferably at least 5 vol. %, more preferably in a range of 10 to 80 vol. %, even more preferably 10 to 55 vol. %, and most preferably in the range of 10 to 40 vol. %.

The inventors found that adjusting the amount of vaporized alcohol by volume of gaseous feed stream to a range of from 10 to 40 vol. % can further improve conversion and yield of the reaction.

For example, the vaporized alcohol obtained in step c) is present in the gaseous feed stream in an amount of from 15 to 35 vol. %. In one specific embodiment, the vaporized alcohol obtained in step c) is present in the gaseous feed stream in an amount of from 15 to 20 vol. % or from 25 to 35 vol. %.

The gaseous feed stream comprising the vaporized alcohol may be purified, dried, or otherwise treated as known in the art, before being fed to step d) of the process according to the invention.

### Step d)

In step d) of the process according to the invention, the vaporized alcohol obtained in step c) is reacted in the presence of the surface-reacted calcium carbonate provided in step b) as a catalyst.

The reaction of the vaporized alcohol in step d) is carried out in the gas phase. Thus, the reaction in step d) can be defined as a gas phase Guerbet self-condensation reaction. As will be understood by a person of skill, this is not to be understood in that the surface-reacted calcium carbonate used as catalyst in step d) is also in the gaseous state. The surface-reacted calcium carbonate used as catalyst in step d) is in the solid state, and may therefore also be defined as heterogeneous catalyst.

The reaction, i.e. the Guerbet self-condensation of the vaporized alcohol obtained in step c), can take place in any reactor which is suitable for gas phase reactions. Non-limiting examples are plug flow reactors and packed-bed or fixed-bed reactors. Technical equipment for carrying out step d) such as, but not-limited to, pre-heater, furnaces, coolers, cooling tanks, back pressure regulator, heat tracing, flow meters, filters etc will be selected by the person of skill as required.

It is possible to carry out the inventive process as a batch process or a continuous process. It is preferred to carry out the process as a continuous process. Hence, in one preferred embodiment, the alcohol is continuously provided in step a), continuously vaporized in step c), and continuously reacted in the presence of the catalyst (SRCC) in step d).

The surface-reacted calcium carbonate provided in step b) may be dried before the reaction with the vaporized alcohol. The drying may be carried out by passing a stream of carrier gas over the surface-reacted calcium carbonate, preferably at elevated temperatures (e.g. in the range of 100 to 500° C. or 300 to 475° C.).

According to one embodiment, the surface-reacted calcium carbonate is dried prior to step d) at a temperature in the range of from 100 to 500° C., preferably 200 to 475° C., and more preferably 300 to 475° C. According to another embodiment, the surface-reacted calcium carbonate used in step d) has a residual total moisture content from 0.01 wt.-% to 0.75 wt.-%, preferably from 0.02 wt.-% to 0.5 wt.-%, based on the total dry weight of the surface-reacted calcium carbonate. According to one preferred embodiment, the surface-reacted calcium carbonate is dried prior to step d) at a temperature in the range of from 100 to 500° C., preferably 200 to 475° C., and more preferably 300 to 475° C., and the surface-reacted calcium carbonate used in step d) has a residual total moisture content from 0.01 wt.-% to 0.75 wt.-%, preferably from 0.02 wt.-% to 0.5 wt.-%, based on the total dry weight of the surface-reacted calcium carbonate.

The reaction in step d) is preferably carried out a defined reaction temperature. According to one embodiment, step d) is carried out at a reaction temperature in the range of from 150 to 500° C., preferably from 150 to 475° C., more preferably from 250 to 475° C., even more preferably from 350 to 475° C., and most preferably from 375 to 475° C.

The inventors have found that a reaction temperature in the range of 375 to 475° C. is particularly advantageous for the process according to the invention.

The process may be defined in terms of its specific weight hourly space velocity (WHSV). The “weight hourly space velocity” as used herein is defined by the weight of the feed, i.e. weight of the feed of alcohol before vaporization in step c), flowing per unit weight of the catalyst (SRCC) per hour.

According to one embodiment, the weight hourly space velocity is at least 2 h⁻¹ (e.g. in a range of 2 to 2000 h⁻¹), and preferably at least 5 h⁻¹ (e.g. 5 to 2000 h⁻¹). In one specific embodiment, the weight hourly space velocity is in a range of 2 to 200 h⁻¹, e.g. from 5 to 100 h⁻¹ or from 10 to 100 h⁻¹.

The reaction in step d) produces an alcohol by condensing two molecules of the alcohol provided in step a) (also referred herein as the “desired product”). The alcohol obtained in step d) contains the sum of the carbon atoms of two molecules of the primary or secondary alcohol, respectively, as provided in step a). For example, if 2-propanol (C3-alcohol) is provided as a secondary alcohol in step a), the alcohol obtained in step d) is 2-methyl-pentan-2-ol (C6-alcohol). If ethanol (C2-alcohol) is provided as primary alcohol in step a), the alcohol obtained in step d) is n-butanol (C4-alcohol), and so on.

The alcohol obtained in step d) may be a linear-chain alcohol or a branched-chain alcohol. In case the alcohol provided in step a) has more than 3 carbon atoms, the alcohol obtained in step d) is preferably a branched-chain alcohol.

Preferably, the alcohol obtained in step d) has a boiling point of below 400° C., more preferably of below 300° C., even more preferably of below 250° C., and most preferably of below 200° C.

The alcohol obtained in step d) can be present in a product mixture with other products of the reaction. For example, the product mixture may contain aldehydes, ethers, esters or other alcohols (e.g. alcohols with a higher molecular weight than the desired product), which are also referred herein as “by-products”. As will be understood by those skilled in the art, the by-products may, for example, be derived from intermediates of the Guerbet reaction (e.g. aldehydes). Each one of the by-products may have its own value, and may be separated and purified individually, if needed.

According to one embodiment, an alcohol is obtained in step d) as part of a product mixture which comprises one or more by-products, such as, but not limited to, alkenes, aldehydes, ethers, esters or other alcohols.

The reaction in step d) may have a specific selectivity for the desired product. The “selectivity” as used herein is the selectivity (S) calculated by

Sⱼ:nⱼ/(nᵢ⁰−nᵢ)*100,
where nᵢ⁰ is the initial amount of C moles (carbon moles) of alcohol before step d), nᵢ is the amount of C moles of unreacted alcohol after step d) and nⱼ is the amount of C moles of product j in the stream of the reaction products.

In one embodiment, the alcohol (desired product) is obtained in step d) with a selectivity of at least 15% (e.g. from 15 to 99.99%), preferably at least 30% (e.g. 30 to 99.99%), more preferably at least 40% (e.g. 40 to 99.99%), and most preferably at least 50% (e.g. 50 to 99.99%).

The vaporized alcohol obtained in step c) may be reacted in step d) in a specific amount, which can be expressed as the conversion or percentage of conversion of the starting material (vaporized alcohol provided in step c)). The “conversion” as used herein is the conversion (X) calculated by

Xᵢ:nᵢ⁰−nᵢ/nᵢ⁰*100,
where nᵢ⁰ is the initial amount of C moles of alcohol before step d) and nᵢ is the amount of C moles of unreacted alcohol after step d).

According to one embodiment, reaction in step d) has a conversion of at least 5% (e.g. from 5 to 99.99%), preferably at least 10% (e.g. 10 to 99.99%), more preferably at least 15% (e.g. 15 to 99.99%), and most preferably at least 20% (e.g. 20 to 99.99%).

The process according to the invention can comprise one or more additional steps such as, but not limited to, condensing the reaction product(s), purifying the reaction product(s), submitting the reaction product(s) to a second reaction, recycling the by-products, recycling the surface-reacted calcium carbonate, reusing the recycled surface-reacted calcium carbonate as a catalyst, etc. The additional process steps can be combined in any order by the skilled person according to her or his needs.

According to one embodiment, the process comprises a step e) of condensing the reaction product(s) obtained in step d), and/or a step f) of purifying the reaction product(s) obtained in step d) or e).

### The Use According to the Invention

In another aspect, the present invention relates to the use a surface-reacted calcium carbonate as a catalyst for a Guerbet self-condensation reaction in the gas phase,
wherein the surface-reacted calcium carbonate is a reaction product of ground natural calcium carbonate (GNCC) or precipitated calcium carbonate (PCC) with carbon dioxide and one or more H₃O⁺ ion donors and wherein the carbon dioxide is formed in situ by the H₃O⁺ ion donors treatment and/or is supplied from an external source, and wherein the surface-reacted calcium carbonate has a specific surface area of at least 15 m²/g, measured using nitrogen and the BET method according to ISO 9277:2010.

It is to be understood that the same embodiments and preferred embodiments of the surface-reacted calcium carbonate provided in step b) of the process according to the invention as disclosed above and herein are also embodiments and preferred embodiments of the surface-reacted calcium carbonate for the use according to the invention. To further illustrate this, specific embodiments and preferred embodiments of the surface-reacted calcium carbonate for the inventive use are repeated in the following.

According to one embodiment, the surface-reacted calcium carbonate for use according to the invention has a volume median particle size (d₅₀) from 1.0 to 75 μm, preferably from 2 to 50 μm, more preferably 3 to 40 μm, even more preferably from 4 to 30 μm, and most preferably from 5 to 15 μm, and/or a top cut (d₉₈) value from 2 to 150 μm, preferably from 4 to 100 μm, more preferably 6 to 80 μm, even more preferably from 8 to 60 μm, and most preferably from 10 to 30 μm.

According to one embodiment, the surface-reacted calcium carbonate for use according to the invention has a specific surface area (BET) from 15 to 200 m²/g, preferably of from 27 to 180 m²/g, more preferably from 30 to 180 m²/g, even more preferably 45 to 180 m²/g, and most preferably from 120 to 180 m²/g, as measured by the BET method.

According to one embodiment, the surface-reacted calcium carbonate for use according to the invention has an intra-particle intruded specific pore volume in the range from 0.10 to 2.3 cm³/g, more preferably from 0.20 to 2.0 cm³/g, even more preferably from 0.40 to 1.8 cm³/g and most preferably from 0.70 to 1.6 cm³/g, calculated from mercury porosimetry measurement.

According to one embodiment, the one or more H₃O⁺ ion donors are selected from the group consisting of hydrochloric acid, sulfuric acid, sulfurous acid, phosphoric acid, citric acid, oxalic acid, an acidic salt, acetic acid, formic acid, and mixtures thereof, preferably selected from the group consisting of hydrochloric acid, sulfuric acid, sulfurous acid, phosphoric acid, oxalic acid, H₂PO₄⁻, being at least partially neutralized by a corresponding cation such as Li⁺, Na⁺ or K⁺, HPO₄²⁻, being at least partially neutralized by a corresponding cation such as Li⁺, Na⁺, K⁺, Mg²⁺, or Ca²⁺ and mixtures thereof, more preferably selected from the group consisting of hydrochloric acid, sulfuric acid, sulfurous acid, phosphoric acid, oxalic acid, or mixtures thereof, and
most preferably the one or more H₃O⁺ ion donor is phosphoric acid.

According to one embodiment, the surface-reacted calcium carbonate for use according to the invention is a reaction product of ground natural calcium carbonate (GNCC) with carbon dioxide and one or more H₃O⁺ ion donors, wherein the one or more H₃O⁺ ion donor is phosphoric acid, and wherein the carbon dioxide is formed in situ by the H₃O⁺ ion donors treatment.

According to one embodiment, the surface-reacted calcium carbonate for use according to the invention has a total number of basic sites from 0.01 to 0.6 mmol/g, preferably from 0.05 to 0.5 mmol/g, more preferably from 0.10 to 0.45 mmol/g, based on the total dry weight of the surface-reacted calcium carbonate, determined by temperature-programmed desorption with carbon dioxide, and/or a total number of acidic sites from 0.01 to 0.6 mmol/g, preferably from 0.05 to 0.5 mmol/g, more preferably from 0.10 to 0.45 mmol/g, based on the total dry weight of the surface-reacted calcium carbonate, determined by temperature-programmed desorption with ammonia.

According to one preferred embodiment of the present invention, the surface-reacted calcium carbonate is used as a catalyst for a Guerbet self-condensation reaction in the gas phase of a primary or secondary alcohol, wherein the primary or secondary alcohol has at least one β-hydrogen.

Further embodiments and preferred embodiments of the primary or secondary alcohol are disclosed above in connection with the process according to the invention. Certain selected embodiments and preferred embodiments are repeated in the following.

According to one embodiment, the surface-reacted calcium carbonate is used as a catalyst for a Guerbet self-condensation reaction of a primary or secondary alcohol having a boiling point of below 200° C., preferably of below 175° C., more preferably below 145° C., and most preferably below 125° C.

According to one embodiment, the surface-reacted calcium carbonate is used as a catalyst for a Guerbet self-condensation reaction of a primary alcohol.

According to one embodiment, the surface-reacted calcium carbonate is used as a catalyst for a Guerbet self-condensation reaction of a primary or secondary alcohol having a branched or linear C₂-C₁₂-alkyl chain, preferably a branched or linear C₂-C₆-alkyl chain.

According to one embodiment, the surface-reacted calcium carbonate is used as a catalyst for a Guerbet self-condensation reaction of an alcohol selected from the group consisting of ethanol, n-propanol, n-butanol, n-pentanol, n-hexanol, n-heptanol, n-octanol, 2-propanol, 2-methyl-1-propanol, 2-butanol, 3-methyl-2-butanol, 2-butanol and 3-pentanol.

### EXAMPLES

### 1. Measurement Methods

In the following, measurement methods implemented in the examples are described.

### Particle Size Distribution

Volume determined median particle size d₅₀ (vol) and the volume determined top cut particle size d₉₈ (vol) was evaluated using a Malvern Mastersizer 3000 Laser Diffraction System (Malvern Instruments Plc., Great Britain). The d₅₀ (vol) or d₉₈ (vol) value indicates a diameter value such that 50% or 98% by volume, respectively, of the particles have a diameter of less than this value. The raw data obtained by the measurement was analyzed using the Mie theory, with a particle refractive index of 1.57 and an absorption index of 0.005. The methods and instruments are known to the skilled person and are commonly used to determine particle size distributions of fillers and pigments. The sample was measured in dry condition without any prior treatment.

The weight determined median particle size d₅₀ (wt) was measured by the sedimentation method, which is an analysis of sedimentation behaviour in a gravimetric field. The measurement was made with a Sedigraph™ 5120 of Micromeritics Instrument Corporation, USA. The method and the instrument are known to the skilled person and are commonly used to determine particle size distributions of fillers and pigments. The measurement was carried out in an aqueous solution of 0.1 wt.-% Na₄P₂O₇. The samples were dispersed using a high-speed stirrer and supersonicated.

### Specific Surface Area (SSA)

The specific surface area was measured via the BET method according to ISO 9277:2010 using nitrogen, following conditioning of the sample by heating at 250° C. for a period of 30 minutes. Prior to such measurements, the sample was filtered within a Buchner funnel, rinsed with deionised water and dried at 110° C. in an oven for at least 12 hours.

### Intra-Particle Intruded Specific Pore Volume (in Cm³/g)

The specific pore volume was measured using a mercury intrusion porosimetry measurement using a Micromeritics Autopore V 9620 mercury porosimeter having a maximum applied pressure of mercury 414 MPa (60 000 psi), equivalent to a Laplace throat diameter of 0.004 μm (˜nm). The equilibration time used at each pressure step was 20 seconds. The sample material was sealed in a 5 cm³ chamber powder penetrometer for analysis. The data were corrected for mercury compression, penetrometer expansion and sample material compression using the software Pore-Comp (Gane, P. A. C., Kettle, J. P., Matthews, G. P. and Ridgway, C. J., “Void Space Structure of Compressible Polymer Spheres and Consolidated Calcium Carbonate Paper-Coating Formulations”, Industrial and Engineering Chemistry Research, 35(5), 1996, p 1753-1764.).

The total pore volume seen in the cumulative intrusion data can be separated into two regions with the intrusion data from 214 μm down to about 1-4 μm showing the coarse packing of the sample between any agglomerate structures contributing strongly. Below these diameters lies the fine inter-particle packing of the particles themselves. If they also have intra-particle pores, then this region appears bi-modal, and by taking the specific pore volume intruded by mercury into pores finer than the modal turning point, i.e. finer than the bi-modal point of inflection, the specific intra-particle pore volume is defined. The sum of these three regions gives the total overall pore volume of the powder, but depends strongly on the original sample compaction/settling of the powder at the coarse pore end of the distribution.

By taking the first derivative of the cumulative intrusion curve the pore size distributions based on equivalent Laplace diameter, inevitably including pore-shielding, are revealed. The differential curves clearly show the coarse agglomerate pore structure region, the inter-particle pore region and the intra-particle pore region, if present. Knowing the intra-particle pore diameter range it is possible to subtract the remainder inter-particle and inter-agglomerate pore volume from the total pore volume to deliver the desired pore volume of the internal pores alone in terms of the pore volume per unit mass (specific pore volume). The same principle of subtraction, of course, applies for isolating any of the other pore size regions of interest.

### Scanning Electron Microscopy (SEM)

The samples were prepared by diluting 50 to 150 μl slurry samples with 5 ml water. The amount of slurry sample depends on solids content, mean value of the particle size and particle size distribution. The diluted samples were filtrated by using a 0.8 μm membrane filter. A finer filter was used when the filtrate is turbid. A doubled-sided conductive adhesive tape was mounted on a SEM stub. This SEM stub was then slightly pressed in the still wet filter cake on the filter. The SEM stub was then sputtered with 8 nm Au. Subsequently, the prepared samples were examined by: a Sigma VP field emission scanning electron microscope (FESEM) (Carl Zeiss AG, Germany) and a variable pressure secondary electron detector (VPSE) and/or secondary electron detector (SE) with a chamber pressure of about 50 Pa. The investigation under the FESEM (Zeiss Sigma VP) was done at 5 kV (Au).

### X-Ray Diffraction (XRD), X-Ray Photoelectron Spectroscopy (XPS), Thermogravimetric Analysis (TGA)

XRD patterns were recorded using a Bruker D2 Phaser powder X-ray diffractometer using Co radiation source, CoKα=1.789 Å. Measurements were carried out between 10-700 2θ using a scan speed of 0.5 s per step. TGA was conducted using a Mettler Toledo TGA/DSC 3+. The samples were heated from 25 up to 600° C. with a ramp of 25° C. and a 10 min hold at 105° C. and 500° C., with an air flow of 80 ml/min. XPS experiments were carried out in a Kratos AXIS Ultra DLD spectrometer using a monochromatic Al Kα radiation (hu=1486.6 eV) operating at 225 W (15 mA, 15 kV). Instrument base pressure was 5×10⁻¹⁰ Torr.

### Further Experimental Techniques

The Ca and P contents of the SRCC solids were prepared by dissolving a sample of the SRCC in aqua regia (a mixture of 1 part per volume of nitric acid (70 wt.-% in water) and 3 parts per volume of hydrochloric acid (35 wt.-% in water)), diluting the obtained solution with water until an about four-fold increase in volume, and analyzing the diluted solution via the inductively coupled plasma optical emission spectroscopy (ICP-OES) technique using a Perkin Elmer Avio 500 device. The Ca and P contents were determined using a calibration curve.

Adsorbed Ammonia and Adsorbed Carbon Dioxide Temperature Programmed Desorption (NH₃-TPD and CO₂-TPD)

The measurements were performed using a Micromeritics ASAP2920 apparatus. 0.1 g of sample was dried in situ under an He flow with a temperature ramp of 5° C. min⁻¹ up to 400° C.

For the NH₃-TPD measurements, the sample was cooled to 100° C. At this point, 20 pulses of 5 cm³ 10 vol.-% NH₃ in He were dosed over the sample (corresponding to an NH₃ flow of 25.3 cm³ min⁻¹).

The sample was then heated to 600° C. with a ramp of 5° C. min⁻¹ to induce desorption of NH₃. The amount of NH₃ desorbed over time was determined using a thermal conductivity detector (TCD). The TCD concentration was plotted over time for the quantitative evaluation and over temperature to determine the temperature position of the desorption peaks. In both cases, a peak deconvolution was performed. To obtain the total amount of desorbed NH₃, a baseline subtraction and full integration of the desorption feature has been performed. Peak deconvolution was performed using the software Fityk.

After obtaining the area under the curve (AUC, A) (from Fityk), the AUC is converted into a quantifiable amount of NH₃ (n_{NH3} in mmol/g) using the below formulae:

Aᵣ=A/100%

V_{NH3,abs}=Aᵣ·V

V_{NH3}=V_{NH3,abs}/mₛₐₘₚₗₑ

m_{NH3}=V_{NH3}·ρ_{NH3}

n_{NH3}=m_{NH3}/M_{NH3}
ρ_{NH3}=0.76 kg/m³, M_{NH3}=17 g/mol
A=obtained Area (%·min), Aᵣ=Area (min), V=Flow 25.2 (cm³/min)
V_{NH3,abs}=absolute amount of desorbed NH₃ (cm³)
V_{NH3}=amount of desorbed NH₃ per g of sample (cm³/g)

For the CO₂-TPD measurements, the sample was cooled to 50° C. and a procedure similar to the one described for NH₃-TPD was employed. The number of basic sites was determined according to the calculation above, using the values ρ_{CO2}=1.98 kg/m³ and M_{CO2}=44.01 g/mol. For calculating the number of acidic or basic sites, it was assumed that only one molecule of NH₃ or CO₂ can adsorb on a single site.

### 2. Materials

### Surface-Reacted Calcium Carbonate (SRCC)

### SRCC1

SRCC1 has d₅₀ (vol)=6.6 μm, a d₉₈ (vol)=13.7 μm, and an intra-particle intruded specific pore volume of 0.939 cm³/g (for the pore diameter range of 0.004 to 0.51 μm).

SRCC1 was obtained by preparing 350 litres of an aqueous suspension of ground calcium carbonate in a mixing vessel by adjusting the solids content of a ground limestone calcium carbonate from Omya SAS, Orgon having a weight based median particle size d₅₀ (wt) of 1.3 μm, as determined by sedimentation, such that a solids content of 10 wt.-%, based on the total weight of the aqueous suspension, is obtained.

Whilst mixing the slurry at a speed of 6.2 m/s, 11.2 kg phosphoric acid was added in form of an aqueous solution containing 30 wt.-% phosphoric acid to said suspension over a period of 20 minutes at a temperature of 70° C. After the addition of the acid, the slurry was stirred for additional 5 minutes, before removing it from the vessel and drying using a jet-dryer.

### SRCC2

SRCC2 has d₅₀ (vol)=5.8 μm, a d₉₈ (vol)=15.4 μm, and an intra-particle intruded specific pore volume of 1.070 cm³/g (for the pore diameter range of 0.004 to 0.34 μm).

SRCC2 was obtained by preparing 10 litres of an aqueous suspension of ground calcium carbonate in a mixing vessel by adjusting the solids content of a ground marble calcium carbonate from Hustadmarmor Norway such that a solids content of 10 wt.-%, based on the total weight of the aqueous suspension, is obtained. The ground calcium carbonate had a weight based particle size distribution of 90% less than 2 μm, as determined by sedimentation. Additionally, a phosphoric acid solution was prepared such that it contained 30% phosphoric acid, based on the total weight of the solution.

Whilst mixing the slurry, 1.8 kg of the phosphoric acid solution was added over 10 minutes. After 20% of the total acid solution was added, 53 g of citric acid anhydride powder was added to the slurry. Throughout the whole experiment the temperature of the suspension was maintained at 70° C.+/−1° C. Finally, after the addition of the acid, the suspension was stirred for additional 5 minutes before removing it from the vessel and allowing it to cool.

### SRCC3

SRCC3 has d₅₀ (vol)=8.3 μm, a d₉₈ (vol)=18.7 μm, and an intra-particle intruded specific pore volume of 1.565 cm³/g (for the pore diameter range of 0.004 to 0.66 μm).

SRCC3 was obtained by preparing 10 litres of an aqueous suspension of ground calcium carbonate in a mixing vessel by adjusting the solids content of a ground marble calcium carbonate from Karabiga, Turkey such that a solids content of 15 wt.-%, based on the total weight of the aqueous suspension, is obtained. The ground calcium carbonate had a weight based median particle size d₅₀ (wt) of 1.4 μm, as determined by sedimentation. In addition, a phosphoric acid solution was prepared such that it contained 30% phosphoric acid, based on the total weight of the solution.

Whilst mixing the slurry, 2.8 kg of the phosphoric acid solution was added over 15 minutes. Throughout the whole experiment the temperature of the suspension was maintained at 70° C.+/−1° C. Finally, after the addition of the acid, the suspension was stirred for additional 5 minutes before removing it from the vessel and allowing it to cool.

### Other Reagents

All commercial reagents were used as received without further purification. Ethanol (technical grade (99.5%) was obtained from VWR chemicals. HAP-H (Hydroxyapatite; 5 μm particle size) was purchased from Sigma-Aldrich. MgO (98%) was obtained from Acros organics.

The properties of the surface-reacted calcium carbonates are shown in Tables 1-3. The properties of the commercially available catalysts are also shown in Table 1.

TABLE 1
BET surface area of SRCC catalysts and comparative catalysts.

| | | BET surface area |
|---|---|---|
| Entry | Catalyst | (m²/g) |
| 1 | SRCC1 | 58.1 |
| 2 | SRCC2 | 160.3 |
| 3 | SRCC3 | 106.7 |
| 4 | MgO | 239.5 |
| 5 | HAP-H* | 100 |

*publicly available information on a commercial sample.

TABLE 2
Bulk and surface composition of the SRCC catalysts.

| | ICP-OES XPS | | | XPS | | |
|---|---|---|---|---|---|---|
| | Ca | P | Ca/P | Ca | P | Ca/P |
| Catalyst | (wt %) | (wt %) | atom ratio | (% at.) | (% at.) | atomic ratio |
| SRCC1 | 38.29 | 7.55 | 3.93 | 13.0 | 6.7 | 1.9 |
| SRCC2 | 40.67 | 6.34 | 4.95 | 14.4 | 7.8 | 1.9 |
| SRCC3 | 37.44 | 12.36 | 2.34 | 14.5 | 8.1 | 1.8 |

TABLE 3
Number of acidic and basic sites of the surface-reacted
calcium carbonates determined by NH₃ and CO₂-TPD.

| | | CO₂-TPD | NH₃-TPD |
|---|---|---|---|
| | | Total number of basic | Total number of acidic |
| Entry | Catalyst | sites (mmol/g) | sites (mmol/g) |
| 1 | SRCC1 | 0.08 | 0.09 |
| 2 | SRCC2 | 0.33 | 0.19 |
| 3 | SRCC3 | 0.08 | 0.12 |

### 3. Examples

### Guerbet Reaction

The Guerbet reaction of ethanol was performed in a continuous U-shaped fixed-bed flow borosilicate reactor (i.d. 8 mm). The liquid feed was pumped by an HPLC pump (LC-20AT, Shimadzu) with a weight hourly space velocity (WHSV) of 4-22 h⁻¹. Mass flow controllers (F-201CV, Bronkhorst) were used to control the flow of N2 carrier gas. Prior to the reaction, the catalyst bed was dried at 400° C. for 2 h under N2 flow (100 mL/min). All catalytic experiments were performed at atmospheric pressure and temperatures of 350-450° C. In a typical experiment, the reactor was loaded with catalysts (0.05-0.3 g) sandwiched between quartz wool plugs. Ethanol was vaporized in N2 and the resulting stream, with 10-64 vol. % of ethanol (23.6 mmol/h), was fed to the reactor at 12.5-200 mL/min. The reaction products were analyzed by on-line GC (Brucker, 430-GC) equipped with FID detector and PoraPLOT Q-HT analytical column. Catalytic activity of the catalysts was characterized by conversion (X), selectivity to products (S), and yield (Y):

Xᵢ:nᵢ⁰−nᵢ/nᵢ⁰*100, Sⱼ:nⱼ/(nᵢ⁰−nᵢ)*100, Yⱼ:Xᵢ·Sⱼ/100,
where nᵢ⁰ is the initial amount of C moles of ethanol, nᵢ is the unreacted C moles of ethanol and nⱼ is the C moles of product j in the stream of the reaction products.

Catalyst performance was evaluated at 400° C. for a time on stream of 3 h. The results are presented in Table 4. Entry 1 shows negligible amount of conversion with only a trace amount of acetaldehyde formation under catalyst free condition. Entries 2 and 3 are the performance shown by the commercially available solid base catalysts such as MgO and hydroxyapatite (HAP-H), respectively. HAP-H showed a better conversion and 1-butanol selectivity compared to MgO. Entries 4 to 6 relate to examples according to the invention using SRCC1 to SRCC3 as catalyst. As can be seen from the results, the conversion of substrate as well as selectivity and yield for 1-butanol in entries 4 to 6 is improved compared to the commercial catalyst MgO. Further, the results for entries 4 to 6 are similar or better than the commercial catalyst HAP-H.

Among all the SRCC catalysts (entries 4-6), SRCC2 showed the best performance with highest 1-butanol yield. In comparison with the commercially available HAP-H, SRCC2 showed better conversion with good butanol yield. Other than the main products 1-butanol and acetaldehyde, minor products that are seen throughout all reactions include various C4 products such as diethyl ether, 1-butanal, ethyl acetate, crotonaldehyde, and the C6 product 1-hexanol.

TABLE 4
Screening of Guerbet reaction of ethanol into butanol using
various catalysts.

| | | | S | Y (%) | | |
|---|---|---|---|---|---|---|
| | | X | 1-butanol | 1- | | ≥C4 |
| Entry | Catalyst | (%) | (%) | butanol | acetaldehyde | products |
| 1 | — | 0.2 | 0 | 0 | 0.2 | 0 |
| 2 | MgO | 5.6 | 23.4 | 1.3 | 3.3 | 0.5 |
| 3 | HAP-H | 13.3 | 51.2 | 6.8 | 3.3 | 3 |
| 4 | SRCC1 | 9.4 | 41.6 | 3.9 | 4.1 | 1 |
| 5 | SRCC2 | 24.3 | 46.3 | 11.3 | 4.2 | 4.7 |
| 6 | SRCC3 | 21.9 | 26.3 | 5.8 | 4.4 | 3.4 |

Reaction conditions: Catalyst amount=0.1 g; WHSV (weight hourly space velocity)=11 h⁻¹; ethanol feed=23.6 mmol/h; ethanol volume=18%; N₂ flow=100 mL/min; temperature=400° C.; time on stream=3 h; HAP-H=Hydroxyapatite (surface area−100 m²/g); catalyst drying=2 h at 400° C. under N₂ flow; X=conversion; S=Selectivity; Y=Yield.

Reaction parameters were optimized with the best catalyst SRCC2 and the results are presented in Table 5. Results are given for an initial time on stream of 3 h and final time of 18 h. Entries 1 to 4 shows the study of catalyst loading, as reflected by the WHSV; ethanol conversion decreased upon decreasing catalyst loading (and increasing WHSV). Entries 3 and 5 to 8 shows the variation of ethanol vol. % in the stream and entries 6, 9 and 10 shows the dependence of reaction temperatures. Overall, the best result of ethanol conversion with good butanol selectivity was obtained when a catalyst loading of 0.1 g (WHSV=11 h⁻¹) with an ethanol vol. of 30% in the stream at a reaction temperature of 400° C.

TABLE 5
Optimization of various reaction parameters for SRCC2 catalyst.

| | Catalyst | | | | | | | | | |
|---|---|---|---|---|---|---|---|---|---|---|
| | Loading | WHSV | Ethanol | Temp. | X (%) | | S (%) | | Y (%) | |
| Entry | (g) | (h⁻¹) | vol. % | (° C.) | 3 h | 18 h | 3 h | 18 h | 3 h | 18 h |
| 1 | 0.3 | 4 | 18 | 400 | 45.8 | 53.4 | 24.5 | 28.2 | 11.2 | 15.1 |
| 2 | 0.2 | 5 | 18 | 400 | 29.3 | 27.3 | 41.1 | 52.1 | 12 | 14.2 |
| 3 | 0.1 | 11 | 18 | 400 | 24.3 | 20.2 | 46.3 | 58.6 | 11.3 | 11.8 |
| 4 | 0.05 | 22 | 18 | 400 | 19.3 | 16.3 | 42.6 | 50 | 8.2 | 8.2 |
| 5 | 0.1 | 11 | 10 | 400 | 35.5 | 27.9 | 30.5 | 39 | 11.5 | 10.9 |
| 6 | 0.1 | 11 | 30 | 400 | 25.6 | 23.1 | 55.3 | 60.8 | 14.2 | 14 |
| 7 | 0.1 | 11 | 47 | 400 | 20.2 | 17.9 | 50 | 65 | 10.1 | 11.6 |
| 8 | 0.1 | 11 | 64 | 400 | 13 | 13 | 56.5 | 68.1 | 7.3 | 10.1 |
| 9 | 0.1 | 11 | 30 | 375 | 9.2 | 8.2 | 66.7 | 74.7 | 6.1 | 6.1 |
| 10 | 0.1 | 11 | 30 | 450 | 56 | 29.7 | 31.1 | 48.7 | 17.4 | 14.4 |

Reaction conditions: ethanol feed=23.6 mmol/h; catalyst drying=2 h at 400° C. under N₂ flow; X=conversion; S=Selectivity of 1-butanol; Y=Yield of 1-butanol.

Detailed comparison of SRCC2's performance with the commercially available HAP-H catalyst is presented in Table 6. Comparison has been carried out in order to check the catalyst stability with respect to the catalyst performance over a prolonged reaction time. Over the course of the reaction SRCC2 catalyst showed better performance than the commercially available HAP-H.

TABLE 6
Comparison between SRCC2 vs HAP-H

| | |
|---|---|
| SHC-3 | HAP-H |

| Entry | Time (h) | X (%) | Y (%) | X (%) | Y (%) |
|---|---|---|---|---|---|
| 1 | 1 | 28.4 | 14.3 | 16.1 | 8.8 |
| 2 | 3 | 25.6 | 14.2 | 13.9 | 7.4 |
| 3 | 6 | 25.6 | 14.5 | 13.6 | 7.3 |
| 4 | 12 | 24.1 | 14.3 | 13.7 | 7.4 |
| 5 | 18 | 23.1 | 14.4 | 13.9 | 7.7 |
| 6 | 24 | 22.3 | 13.9 | 13.7 | 7.7 |
| 7 | 30 | 22.1 | 13.9 | 12.8 | 7 |
| 8 | 36 | 21 | 13.2 | 12.5 | 7.1 |
| 9 | 42 | 20.7 | 13.2 | 11.8 | 7.1 |
| 10 | 48 | 20.1 | 12.9 | 11.7 | 7 |
| 11 | 54 | 20.6 | 13.2 | 11.9 | 7.4 |
| 12 | 60 | 20.7 | 13.3 | 11.3 | 7.2 |
| 13 | 66 | 19.8 | 12.9 | 11.5 | 7.3 |

Reaction conditions: Catalyst amount=0.1 g; WHSV (weight hourly space velocity)=11 h⁻¹; Ethanol feed=23.6 mmol/h; Ethanol volume=30%; N₂ flow=50 mL/min; Temperature=400° C.; HAP-H=Hydroxyapatite (surface area−100 m²/g); catalyst drying=2 h at 400° C. under N₂ flow; X=conversion; Y=Yield of butanol.

1. A process for preparing an alcohol by a Guerbet self-condensation reaction in the gas phase, comprising the steps of:
a) providing a primary or secondary alcohol, wherein the primary or secondary alcohol has at least one β-hydrogen;
b) providing a surface-reacted calcium carbonate, wherein the surface-reacted calcium carbonate is a reaction product of ground natural calcium carbonate (GNCC) or precipitated calcium carbonate (PCC) with carbon dioxide and one or more H₃O⁺ ion donors and wherein the carbon dioxide is formed in situ by the H₃O⁺ ion donors treatment and/or is supplied from an external source, and wherein the surface-reacted calcium carbonate has a specific surface area of at least 15 m²/g, measured using nitrogen and the BET method according to ISO 9277:2010;
c) vaporizing the alcohol provided in step a);
d) reacting the vaporized alcohol obtained in step c) in the presence of the surface-reacted calcium carbonate provided in step b) as a catalyst.

2. The process according to claim 1, wherein the primary or secondary alcohol provided in step a) has a boiling point of below 200° C., preferably of below 175° C., more preferably below 145° C., and most preferably below 125° C.

3. The process according to claim 1, wherein the alcohol provided in step a) is a primary alcohol.

4. The process according to claim 1, wherein the primary or secondary alcohol provided in step a) is a primary or secondary alcohol having a branched or linear C₂-C₁₂-alkyl chain, preferably a branched or linear C₂-C₆-alkyl chain, or
wherein the primary or secondary alcohol provided in step a) is the selected from the group consisting of ethanol, n-propanol, n-butanol, n-pentanol, n-hexanol, n-heptanol, n-octanol, 2-propanol, 2-methyl-1-propanol, 2-butanol, 3-methyl-2-butanol, 2-butanol and 3-pentanol.

5. The process according to claim 1, wherein the surface-reacted calcium carbonate provided in step b) has
a volume median particle size (d₅₀) from 1.0 to 75 μm, preferably from 2 to 50 μm, more preferably 3 to 40 μm, even more preferably from 4 to 30 μm, and most preferably from 5 to 15 μm, and/or
a top cut (d₉₈) value from 2 to 150 μm, preferably from 4 to 100 μm, more preferably 6 to 80 μm, even more preferably from 8 to 60 μm, and most preferably from 10 to 30 μm.

6. The process according to claim 1, wherein the surface-reacted calcium carbonate provided in step b) has a specific surface area (BET) from 15 to 200 m²/g, preferably of from 27 to 180 m²/g, more preferably from 30 to 180 m²/g, even more preferably 45 to 180 m²/g, and most preferably from 120 to 180 m²/g, as measured by the BET method.

7. The process according to claim 1, wherein the surface-reacted calcium carbonate provided in step b) has an intra-particle intruded specific pore volume in the range from 0.10 to 2.3 cm³/g, more preferably from 0.20 to 2.0 cm³/g, even more preferably from 0.40 to 1.8 cm³/g and most preferably from 0.70 to 1.6 cm³/g, calculated from mercury porosimetry measurement.

8. The process according to claim 1, wherein the one or more H₃O⁺ ion donors are selected from the group consisting of hydrochloric acid, sulfuric acid, sulfurous acid, phosphoric acid, citric acid, oxalic acid, an acidic salt, acetic acid, formic acid, and mixtures thereof,
preferably selected from the group consisting of hydrochloric acid, sulfuric acid, sulfurous acid, phosphoric acid, oxalic acid, H₂PO₄⁻, being at least partially neutralized by a corresponding cation such as Li⁺, Na⁺ or K⁺, HPO₄²⁻, being at least partially neutralized by a corresponding cation such as Li⁺, Na⁺, K⁺, Mg²⁺, or Ca²⁺ and mixtures thereof,
more preferably selected from the group consisting of hydrochloric acid, sulfuric acid, sulfurous acid, phosphoric acid, oxalic acid, or mixtures thereof, and
most preferably the one or more H₃O⁺ ion donor is phosphoric acid.

9. The process according to claim 1, wherein the surface-reacted calcium carbonate is a reaction product of ground natural calcium carbonate (GNCC) with carbon dioxide and one or more H₃O⁺ ion donors, wherein the one or more H₃O⁺ ion donor is phosphoric acid, and wherein the carbon dioxide is formed in situ by the H₃O⁺ ion donors treatment.

10. The process according to claim 1, wherein the surface-reacted calcium carbonate provided in step b) has
a total number of basic sites from 0.01 to 0.6 mmol/g, preferably from 0.05 to 0.5 mmol/g, more preferably from 0.10 to 0.45 mmol/g, based on the total dry weight of the surface-reacted calcium carbonate, determined by temperature-programmed desorption with carbon dioxide, and/or
a total number of acidic sites from 0.01 to 0.6 mmol/g, preferably from 0.05 to 0.5 mmol/g, more preferably from 0.10 to 0.45 mmol/g, based on the total dry weight of the surface-reacted calcium carbonate, determined by temperature-programmed desorption with ammonia.

11. The process according to claim 1, wherein the surface-reacted calcium carbonate is dried prior to step d) at a temperature in the range of from 100 to 500° C., preferably from 300 to 475° C., and/or
wherein the surface-reacted calcium carbonate used in step d) has a residual total moisture content from 0.01 wt.-% to 0.75 wt.-%, preferably from 0.02 wt.-% to 0.5 wt.-%, based on the total dry weight of the surface-reacted calcium carbonate.

12. The process according to claim 1, wherein the vaporized alcohol obtained in step c) is present in a gaseous feed stream in an amount of at least 2 vol. %, preferably in a range of 10 to 80 vol. %, more preferably 10 to 55 vol. %, and most preferably in the range of 10 to 40 vol. %, and/or
wherein the vaporized alcohol obtained in step c) is mixed with a carrier gas selected from the group consisting of helium, nitrogen, argon, hydrogen and mixtures thereof, preferably nitrogen.

13. The process according to claim 1, wherein step d) is carried out at a reaction temperature in the range of from 150 to 500° C., preferably from 150 to 475° C., more preferably from 250 to 475° C., and most preferably from above 375 to 475° C.

14. A method of catalyzing a Guerbet self-condensation reaction in the gas phase, comprising providing a catalyst comprising a surface-reacted calcium carbonate,
wherein the surface-reacted calcium carbonate is a reaction product of ground natural calcium carbonate (GNCC) or precipitated calcium carbonate (PCC) with carbon dioxide and one or more H₃O⁺ ion donors and wherein the carbon dioxide is formed in situ by the H₃O⁺ ion donors treatment and/or is supplied from an external source, and wherein the surface-reacted calcium carbonate has a specific surface area of at least 15 m²/g, measured using nitrogen and the BET method according to ISO 9277:2010.

15. The method according to claim 14, wherein the Guerbet self-condensation reaction is a reaction of a primary or secondary alcohol having at least one β-hydrogen.
